(19) United States
(12) Patent Application Publication
(10) Pub. No.: US 2014/0027330 A1
(43) Pub. Date: Jan. 30, 2014

(54) SURGICAL TRAY PROTECTION SYSTEM AND METHOD

(71) Applicant: Richard M. Lavelle, Wexford, PA (US)

(72) Inventor: Richard M. Lavelle, Wexford, PA (US)

(21) Appl. No.: 13/954,207

(22) Filed: Jul. 30, 2013

Related U.S. Application Data
(60) Provisional application No. 61/677,130, filed on Jul. 30, 2012.

(51) Int. Cl. A61B 19/02; B65B 11/00

(52) U.S. Cl. CPC A61B 19/0271; B65B 11/00
USPC 206/370; 206/497; 53/461

(57) ABSTRACT

A protection system and method for use with surgical trays and to prevent a sterilization wrap around a sterilized surgical tray from being ripped, punctured, or torn is disclosed. The system includes at least one corner protector positioned on a respective corner of a surgical tray, and at least one edge guard positioned on a respective edge of the surgical tray. The at least one corner protector and at least one edge guard provide a cushion between the surgical tray and a sterilization wrap to prevent the wrap from being ripped, punctured, or torn.

### TECHNICAL FIELD AND BACKGROUND OF THE INVENTION

The present invention relates to the field of protection systems. In particular, the invention relates to a protection system and method for use with surgical trays.

Surgical trays are used to organize, sterilize, and carry surgical instruments into an operating room for use by a surgeon. Because the surgical trays and instruments carry pathogens, the trays and instruments must be sterilized prior to being placed in the operating room. This is done by placing the instruments in the trays, wrapping the trays in sterile wraps, as shown in FIG. 1, and placing the wrapped trays in a sterilization chamber where a sterilization agent such as steam, gas, or plasma is used to sterilize the trays and instruments. The wrapped trays are then stored for later use or carried to the operating room for immediate use.

Unfortunately, after the sterilization process has taken place, it is often discovered that the feet and sharp corners of the trays have punctured through the sterile wrap or that the wrap has been ripped when removing the wrapped trays from storage shelves, metal stands or wire racks, thereby causing the trays and instruments contained therein to be contaminated. If a surgical instrument tray and the instruments therein cannot be used because the sterilization wrap has been ripped, punctured or torn, the surgery might have to be delayed or possibly canceled, costing the hospital or surgery center time, money and frustration for both the staff and physicians.

### SUMMARY OF THE INVENTION

Accordingly, the above-noted shortcomings of the prior art are addressed by the present invention, which provides a surgical tray protection system that prevents the wrap from being ripped, punctured or torn, while allowing circulation of the sterilization agent between the tray and the wrap. In addition, this method allows for easier movement by sliding of the tray. This is accomplished by allowing all of the weight to be absorbed on the corner protector and edge protector.

According to one aspect of the invention, a surgical tray protection system to prevent a sterilization wrap around a sterilized surgical tray from being ripped, punctured, or torn, includes at least one corner protector positioned on a respective corner of a surgical tray, and at least one edge guard positioned on a respective edge of the surgical tray. The at least one corner protector and at least one edge guard provide a cushion between the surgical tray and a sterilization wrap to prevent the wrap from being ripped, punctured, or torn.

According to another aspect of the invention, a surgical tray protection system to prevent a sterilization wrap around a sterilized surgical tray from being ripped, punctured, or torn, includes a plurality of corner protectors for being positioned on respective corners of a surgical tray and a plurality of edge guards for being positioned on respective edges of the surgical tray between corners of the surgical tray to provide a cushion between the surgical tray and a sterilization wrap to prevent the wrap from being ripped, punctured, or torn. Each of the plurality of corner protectors include a base and first and second sides. The first and second sides extend upwardly from a top of the base and are connected at proximal ends to define a corner. Each of the sides includes a bottom edge and a top edge, wherein a first portion of each bottom edge is connected to the base and a second portion of each bottom edge extends beyond the base such that a distal end of each side is disposed beyond the base and free to flex independently from the base. In addition, the top edges define an open area therebetween free of reinforcement structures to permit a portion of the top edges to move relative to the base.

According to another aspect of the invention, a method of protecting a sterilization wrap wrapped around a sterilized surgical tray includes the step of providing a surgical tray protection system having a plurality of corner protectors and a plurality of edge guards. The method further including the steps of positioning one of the plurality of corner protectors at each bottom corner of the surgical tray, positioning one of the plurality of edge guards at each bottom edge of the surgical tray between each of the bottom corners, and wrapping the surgical tray with a sterilization wrap.

### BRIEF DESCRIPTION OF THE DRAWINGS

The invention may be best understood by reference to the following description in conjunction with the accompanying drawing figures in which:

FIG. 1 shows a wrapped surgical tray without any protection system;

FIG. 2 shows a surgical tray protection system according to an embodiment of the invention installed on a surgical tray;

FIG. 3 shows the surgical tray protection system of FIG. 2 installed on a surgical tray;

FIG. 4 shows a corner protector of the system of FIG. 2 placed between a corner foot of a surgical tray and a sterile wrap;

FIG. 5 is a perspective view of a corner protector of the system of FIG. 2;

FIG. 6 is a top view of the corner protector of FIG. 5;

FIG. 7 is a bottom view of the corner protector of FIG. 5;

FIG. 8 is a right side view of the corner protector of FIG. 5;

FIG. 9 is a right, rear side view of the corner protector of FIG. 5;

FIG. 10 is a left, rear side view of the corner protector of FIG. 5;

FIG. 11 is a left side view of the corner protector of FIG. 5;

FIG. 12 is a perspective view of a corner protector of the system of FIG. 2; and

FIG. 13 shows edge guards of the system of FIG. 2.

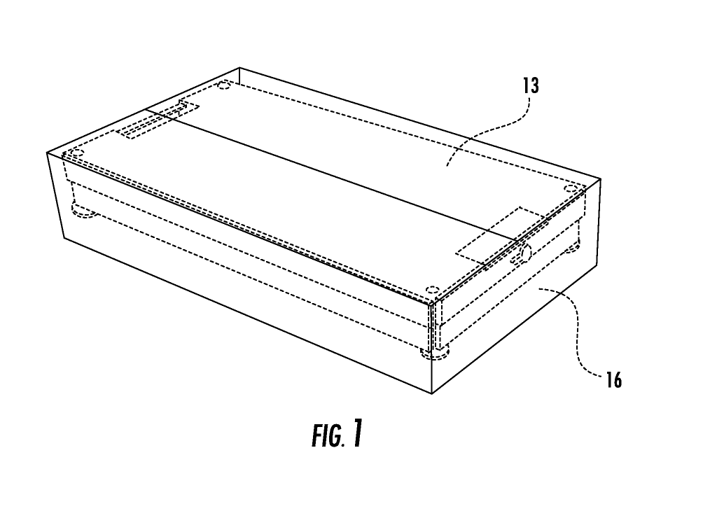

### DESCRIPTION OF THE PREFERRED EMBODIMENT AND BEST MODE

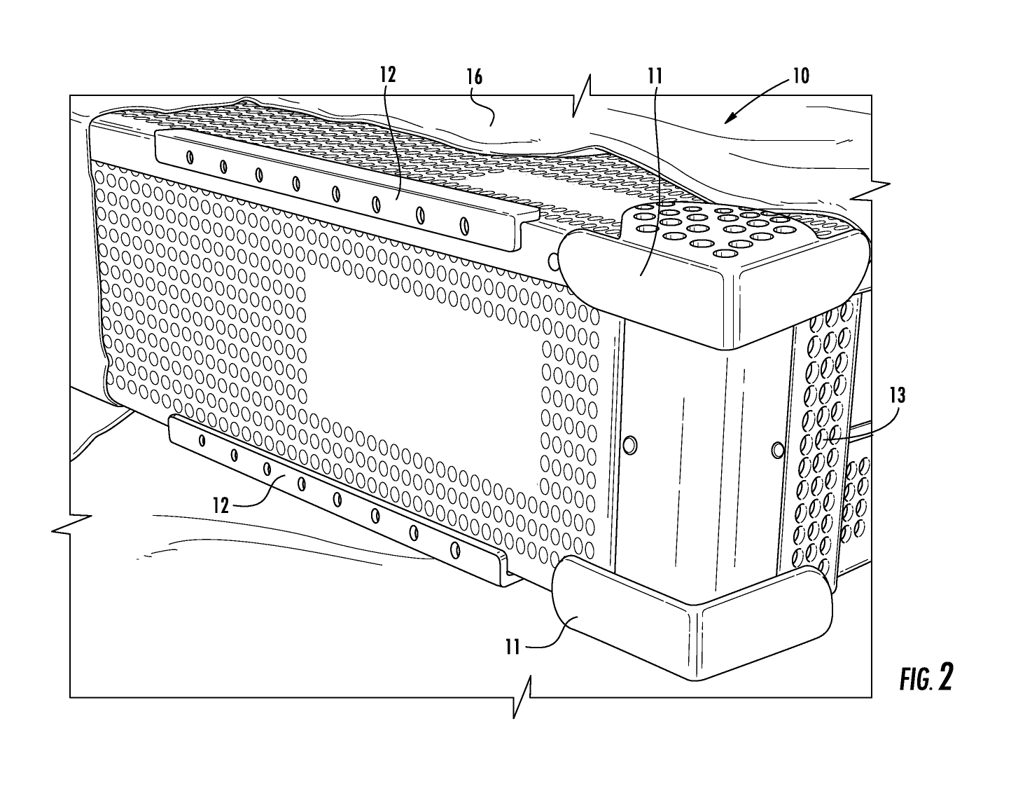

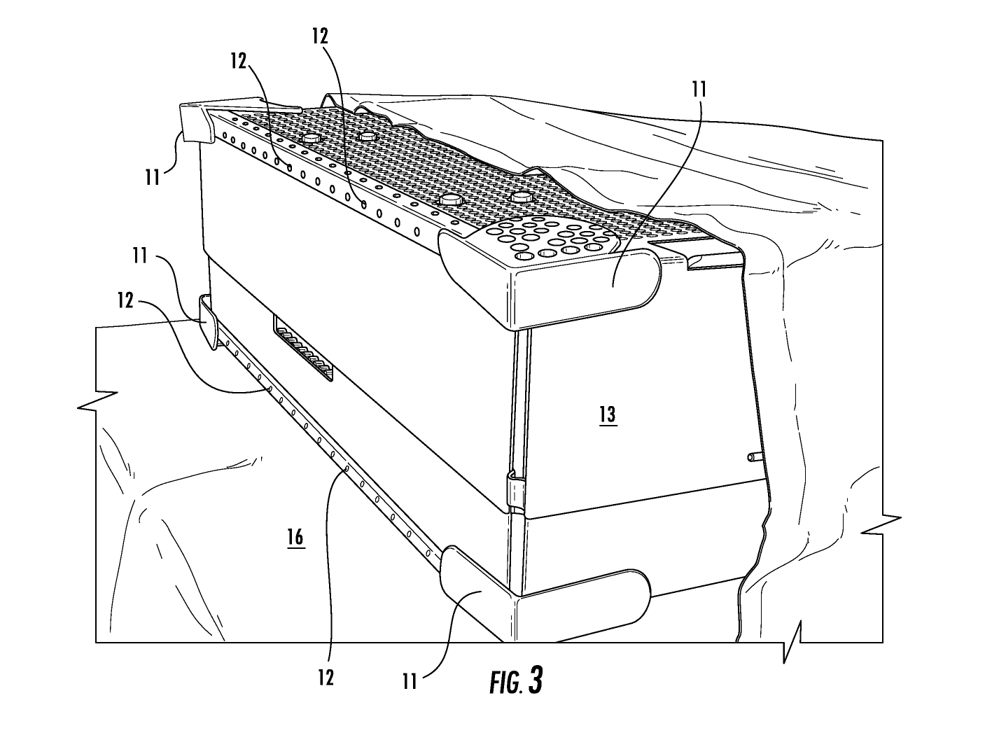

Referring now specifically to the drawings, a surgical tray protection system according to an embodiment of the invention is illustrated in FIGS. 2 and 3 and shown generally at reference numeral 10. The system 10 includes a plurality of corner protectors 11 and edge guards 12 positioned around a surgical tray 13. As shown, a corner protector 11 is positioned at each corner of the tray 13 and an edge guard 12 is positioned along each edge of the tray 13 between the corner protectors 11. The system 10 may be used on rounded corners and edges, FIG. 2, or on sharp, squared corners and edges, FIG. 3.

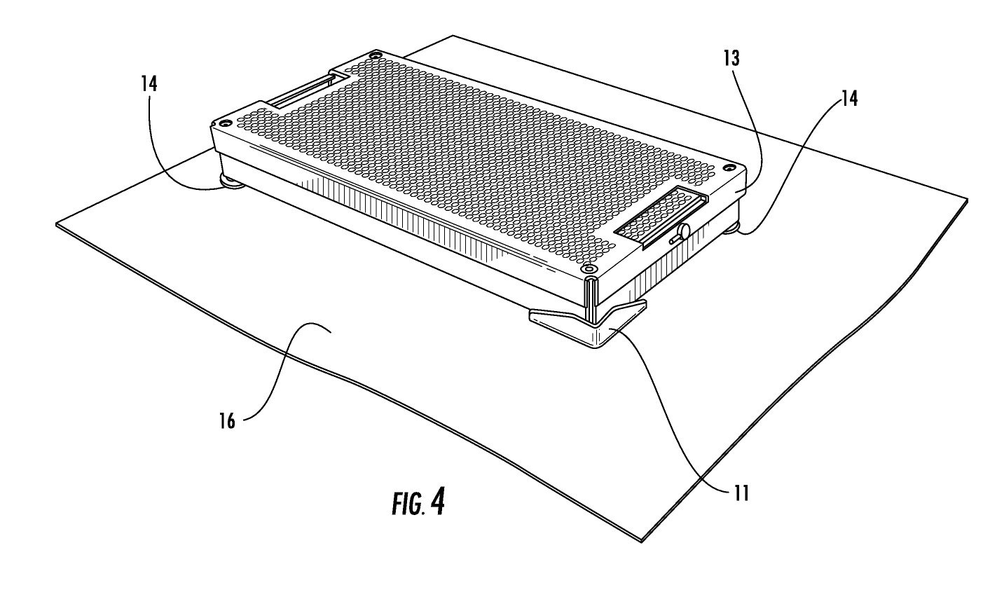

The corner protectors 11 are designed to be placed on a corner of the surgical tray 13 between a foot 14 of the tray 13 and a sterile wrap 16, see FIG. 4. The corner protectors 11 prevent the foot 14 and corners from puncturing through the sterile wrap 16 while allowing a sterilization agent to circulate around the foot 14 and between the sterile wrap 16 and corner protectors 11. In the event that the surgical tray 13 does not include feet, the corner protectors 11 are placed at each corner between the tray 13 and wrap 16 to prevent sharp edges of the tray 13 from ripping the wrap 16, see FIGS. 2 and 3.

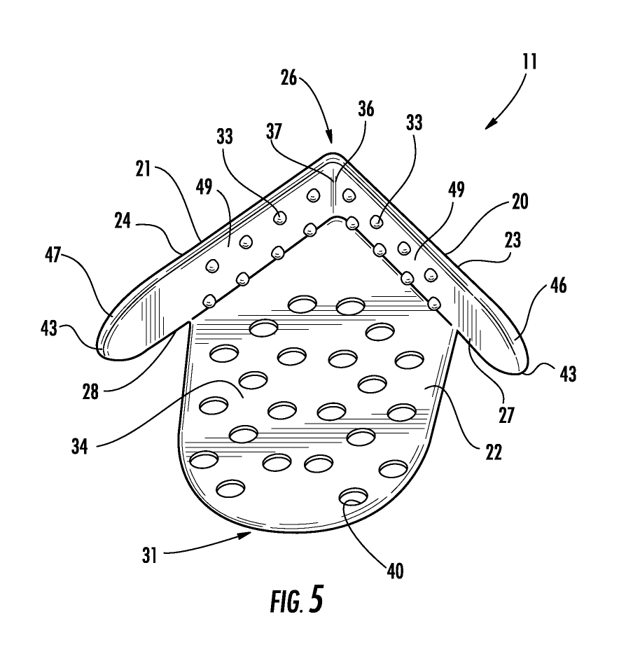

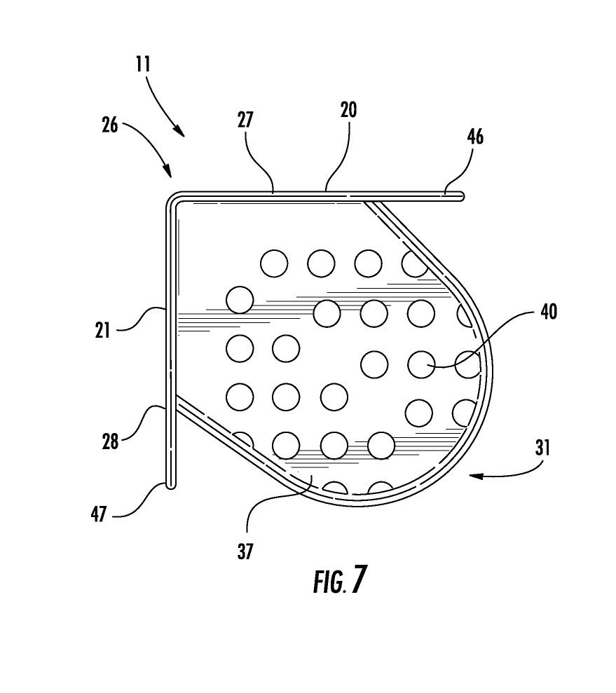

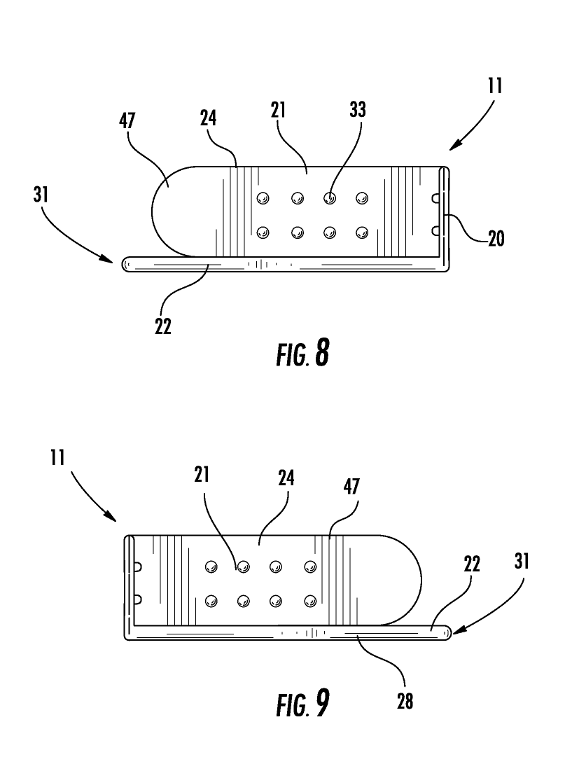

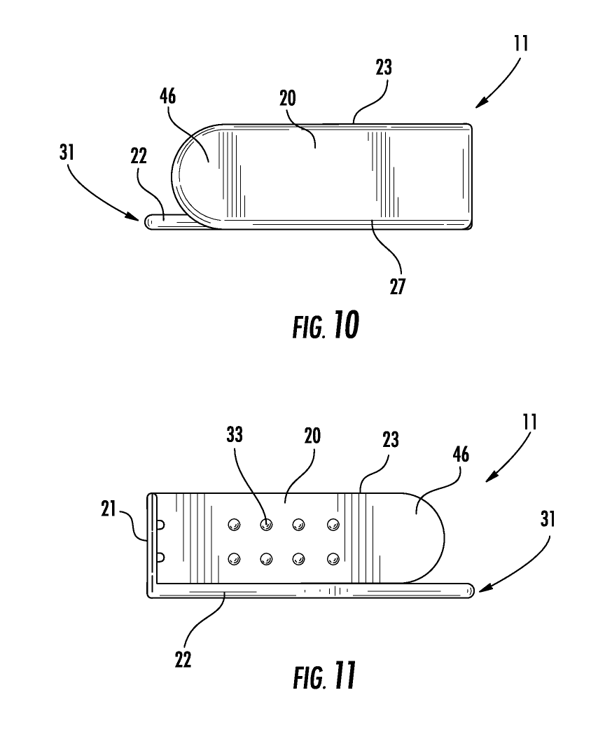

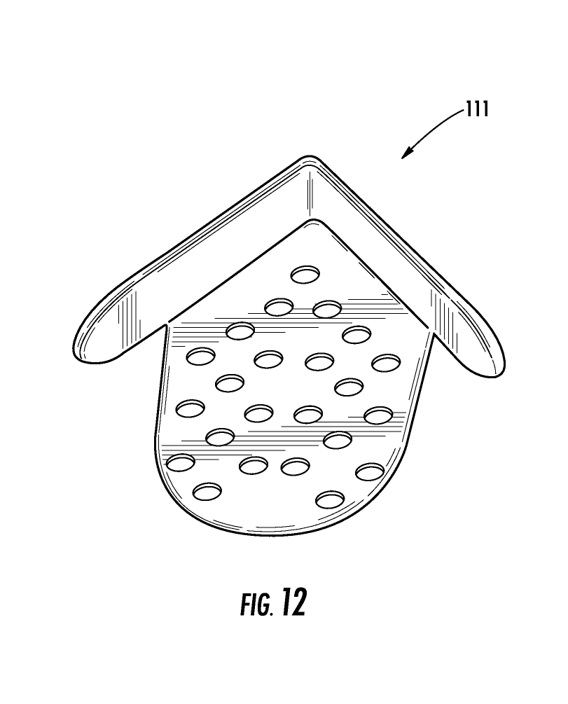

Referring to FIGS. 5-11, the corner protectors 11 each include sides 20 and 21, a base 22, a corner 26 and a front 31, and apertures 40. The base 22 is a smooth, non-sloping base with a rounded front 31. As shown, the base 22 has a planar top 34 and a planar bottom 37, which allows the tray 13 to rest evenly on the base 22 and the base 22 to rest evenly on the wrap 16. The smoothness of the top and bottom 34 and 37 prevents the corner protectors 11 from sticking to the sterile wrap 16 during and after sterilization. The sides 20 and 21 of the corner protector 11 are also smooth and have rounded ends 43 to prevent sticking to the wrap 16.

The sides 20 and 21 each include spaced-apart, parallel top 23, 24 and bottom 27, 28 edges, respectively, extending between proximal ends 36, 37 and free distal ends 46 and 47. The sides 20 and 21 are connected at the proximal ends 36, 37 to form corner 26. As shown, a first portion of the bottom edges 27, 28 are connected to the base 22 and a second portion of the bottom edges 27, 28 extend past the rounded front 31 of the base 22 to create the free distal ends 46 and 47 which are capable of flexing inwardly towards a center of the protector 11 or outwardly away from the center of the protector 11. In addition, the top edges 23, 24 define an area therebetween free of reinforcement structures to accept the tray 13 therebetween and to permit a portion of the top edges 23, 24 to move relative to the base 22 (i.e., inwardly/outwardly).

This arrangement allows the protector 11 to be used with both rounded edge surgical trays and straight edge surgical trays. When used with a rounded tray, the free distal ends 46 and 47 flex outwardly to allow the rounded tray to be positioned farther back into the protector 11, and thus, the tray may be securely positioned on the base 22 at about its center instead of its edge. When used with a square corner tray, the free ends remain in their natural, non-use position and extend along the sides of the tray.

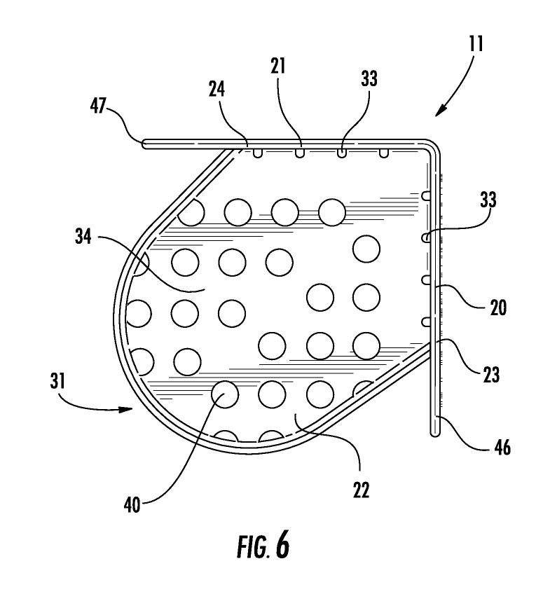

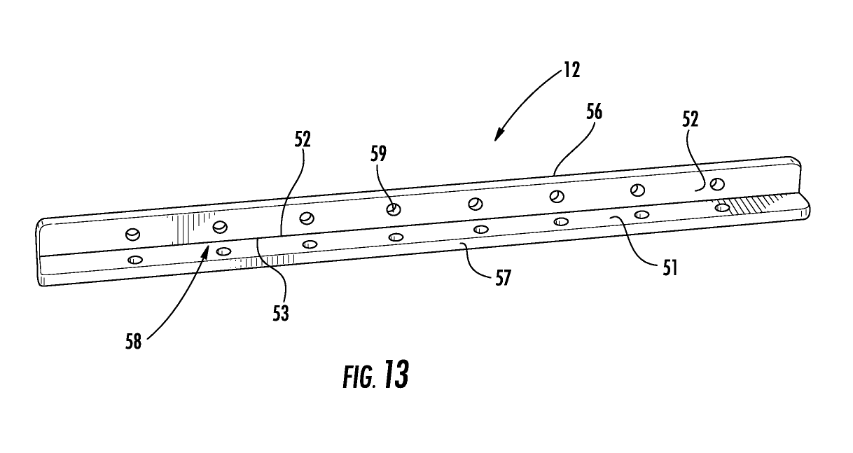

As shown in FIGS. 5 and 6, studs 33 are positioned on an inner surface 49 of the sides 20 and 21. The studs 33 allow the protector 11 to be positioned against the tray 13 without being flush against the tray 13. Thus, the studs 33 create channels between the tray 13 and the sides 20 and 21 of the protector 11. These channels allow the sterilizing agent to flow into the corners of the tray 13 and along its sides between the protector 11 and the tray 13, thereby sterilizing the entire tray 13. Studs 33 may also be placed on the base 22 or may be eliminated altogether, FIG. 12—reference number 111, to provide smooth inner and top surfaces on the sides and base.

A plurality of apertures 40 are formed through the base 22 to allow the sterilization agent to circulate easily between the wrap 16 and the corner protector 11, the corner protector 11 and the surgical tray 13, and the wrap 16 and the surgical tray 13. The apertures 40 prevent the base 22 from inhibiting the circulation of the sterilization agent. Additionally, the apertures 40 help enhance drying times.

As shown in FIG. 13, the edge guards 12 are elongated L-shaped or angled strips formed by first and second walls 50 and 51 disposed substantially perpendicular to each other. The walls 50 and 51 each have proximal ends 52, 53 and free distal ends 56, 57, respectively. The walls 50 and 51 are connected together at the proximal ends 52 and 53 to form an elongated corner 58 and the free distal ends 56, 57 are free to move inwardly and outwardly from the corner 58 to allow the edge guards 12 to accept surgical tray edges that are rounded or squared, and to accept edges that are formed from two tray walls that are slightly off perpendicular. Each of the walls 50 and 51 have apertures 59 to allow the sterilization agent to circulate easily between the wrap 16 and the edge guards 12, the edge guards 12 and the surgical tray 13, and the wrap 16 and the surgical tray 13. The apertures 59 prevent the edge guards 12 from inhibiting the circulation of the sterilization agent and help enhance drying times.

The corner protectors 11 and the edge guards 12 are made of a medical grade material that does not retain moisture, such as silicone rubber. During the sterilization process of moisture absorbing products, moisture can sometimes remain after the drying period, thereby increasing the ability of water borne pathogens to develop and contaminate the contents of the surgical tray 13. By using a non-moisture absorbing material, water borne pathogens can be prevented. The material also provides a “tackiness” to help keep the corner protectors 11 and edge guards 12 in place on the tray 13, and allows the corner protectors 11 and edge guards 12 to be cut to size for a desired application and disposed of after a single use.

In use, the surgical tray 13 is filled with instruments to be sterilized. A corner protector 11 is placed at each corner of the surgical tray 13 and one or more edge guards 12 are placed along each edge of the surgical tray 13 (the number of edge guards is dependent on the length of the surgical tray 13). It should be appreciated that while the system 10 is shown with corner protectors 11 and edge guards 12 positioned along both top and bottom corners and edges of the surgical tray 13, a minimal protection system 10 may be used by only placing corner protectors 11 and edge guards 12 along the bottom corners and edges of the tray 13—this would provide protection from puncturing and tearing due to feet or sliding of the tray 13 along a rack or shelf.

Once the corner protectors 11 and edge guards 12 are installed on the tray 13, the tray 13 is wrapped with a sterilization wrap 16 and placed in a sterilization chamber for sterilizing. Once the sterilization is finished, the wrapped tray 13 is removed from the sterilization chamber and either taken to surgery or placed on a storage shelf or rack for later use.

Often times (particularly when the wrapped tray 13 is placed on a storage shelf or rack), personnel have to move the trays 13. These trays can be heavy and as a result, the wrapped trays 13 are slid into a desired position. The edge guards 12 aid in this sliding movement of the tray by elevating the weight of the tray 13 off of the sterilization wrap 16 underneath. This results in easier movement of the tray 13 and a reduction in holes, rips, and tears occurring to the wrap 16. The use of the corner protectors 11 further elevates the tray 13 to aid in sliding.

Additionally, when the system 10 is used, a cushion effect is created when the surgical tray 13 is lowered onto a surface or support (e.g. front of a heavy tray 13 is lowered and then followed by a back of the tray 13 being lowered into a surgical (sterile) field). Also, trays are often stacked one on top of another to preserve space requirements. By using the system 10 on both the top and bottom of trays 13 being stacked vertically, the system 10 provides a cushion effect by elevating the trays 13 and eliminating metal to metal contact between trays; thus, reducing holes, rips, and tears in the wraps 16.

A surgical tray protection system and method is described above. Various details of the invention may be changed without departing from its scope. Furthermore, the foregoing description of the preferred embodiments of the invention and best mode for practicing the invention are provided for the purpose of illustration only and not for the purpose of limitation.

I claim:

1. A surgical tray protection system to prevent a sterilization wrap around a sterilized surgical tray from being ripped, punctured, or torn, comprising:
(a) at least one corner protector positioned on a respective corner of a surgical tray;
(b) at least one edge guard positioned on a respective edge of the surgical tray; and
(c) wherein the at least one corner protector and at least one edge guard provide a cushion between the surgical tray and a sterilization wrap to prevent the wrap from being ripped, punctured, or torn.

2. The surgical tray protection system according to claim 1, wherein the system includes four corner protectors, wherein each of the corner protectors is positioned on a respective bottom corner of the surgical tray to prevent sharp corners or feet positioned at each corner from ripping, puncturing, or tearing the sterilization wrap.

3. The surgical tray protection system according to claim 1, wherein the system includes eight corner protectors, wherein each of the corner protectors is positioned on respective bottom and top corners of the surgical tray, the corner protectors on the bottom corners of the tray being adapted to prevent sharp corners or feet positioned at each of the corners from ripping, puncturing, or tearing the sterilization wrap and the corner protectors on the top corners being adapted to prevent sharp corners from ripping, puncturing, or tearing the sterilization wrap and to provide a cushion between stacked surgical trays.

4. The surgical tray protection system according to claim 2, wherein at least one edge guard is positioned along an edge between each of the bottom corners to prevent ripping, puncturing, or tearing of the sterilization wrap.

5. The surgical tray protection system according to claim 3, wherein at least one edge guard is positioned along an edge between each of the bottom corners to prevent ripping, puncturing, or tearing of the sterilization wrap and between each of the top corners to prevent ripping, puncturing, or tearing of the sterilization wrap and to provide a cushion between stacked surgical trays.

6. The surgical tray protection system according to claim 1, wherein the at least one corner protector includes:
(a) a base having a front end and an opposed back end, the back end having first and second edges defining a V-shape;
(b) first and second sides, each of the sides having spaced-apart, parallel top and bottom edges extending between a proximal end and a distal end, the sides being connected at the proximal ends to form a corner;
(c) wherein a first portion of the bottom edge of the first side adjacent the proximal end of the first side is joined to the first edge of the base and a second portion of the bottom edge of the first side extends beyond the base such that the distal end of the first side is free to flex independently from the base;
(d) wherein a first portion of the bottom edge of the second side adjacent the proximal end of the second side is joined to the second edge of the base and a second portion of the bottom edge of the second side extends beyond the base such that the distal end of the second side is free to flex independently from the base; and
(e) wherein the top edges define an open area therebetween free of reinforcement structures to permit a portion of the top edges to move relative to the base.

7. The surgical tray protection system according to claim 1, wherein the at least one corner protector includes:
(a) a base;
(b) first and second sides extending upwardly from a top of the base, each of the sides having a bottom edge, wherein a first portion of each bottom edge is connected to the base and a second portion of each bottom edge extends beyond the base such that a distal end of each side is disposed beyond the base; and
(c) wherein proximal ends of the first and second sides are joined to each other so as to define a corner, the first and second sides cooperate with the base to define three mutually perpendicular planes that define an open area configured to receive a corner of a surgical tray having a side wall with a height substantially greater than the first and second sides of the corner protector therein.

8. The surgical tray protection system according to claim 1, wherein the at least one edge guard includes:
(a) a first side having a proximal end and a distal end;
(b) a second side having a proximal end and a distal end; and
(c) wherein the proximal ends of the first and second sides are connected together such that the first and second sides are substantially perpendicular to each other to form an elongated strip adapted to conform to an edge of a surgical tray.

9. The surgical tray protection system according to claim 8, wherein the distal ends of the first and second sides are free to move inwardly and outwardly to allow the at least one edge guard to conform to an edge of a surgical tray that is non-perpendicular.

10. The surgical tray protection system according to claim 1, wherein the at least one corner protector and at least one edge guard include apertures to permit a sterilization agent to circulate therethrough.

11. A surgical tray protection system to prevent a sterilization wrap around a sterilized surgical tray from being ripped, punctured, or torn, comprising:
(a) a plurality of corner protectors for being positioned on respective corners of a surgical tray, each of the plurality of corner protectors having:
(i) a base; and
(ii) first and second sides extending upwardly from a top of the base and connected at proximal ends to define a corner, each of the sides having a bottom edge and a top edge, wherein a first portion of each bottom edge is connected to the base and a second portion of each bottom edge extends beyond the base such that a distal end of each side is disposed beyond the base and free to flex independently from the base, and wherein the top edges define an open area therebetween free of reinforcement structures to permit a portion of the top edges to move relative to the base; and
(b) a plurality of edge guards for being positioned on respective edges of the surgical tray between corners of the surgical tray to provide a cushion between the surgical tray and a sterilization wrap to prevent the wrap from being ripped, punctured, or torn.

12. The surgical tray protection system according to claim 11, wherein each of the plurality of edge guards includes:
(a) first and second sides joined together such that the first and second sides are substantially perpendicular to each other; and
(b) a plurality of apertures extending through the first and second sides to permit a sterilization agent to circulate therethrough.

13. The surgical tray protection system according to claim 12, wherein the first and second sides are joined at their proximal ends to form an elongated corner such that distal ends of the first and second sides are permitted to move inwardly and outwardly from the corner.

14. A method of protecting a sterilization wrap wrapped around a sterilized surgical tray, comprising the steps of:
(a) providing a surgical tray protection system, having:
(i) a plurality of corner protectors; and
(ii) a plurality of edge guards;
(b) positioning one of the plurality of corner protectors at each bottom corner of the surgical tray;
(c) positioning one of the plurality of edge guards at each bottom edge of the surgical tray between each of the bottom corners; and
(d) wrapping the surgical tray with a sterilization wrap.

15. The method according to claim 14, further including the steps of:
(a) positioning one of the plurality of corner protectors at each top corner of the surgical tray; and
(b) positioning one of the plurality of edge guards at each top edge of the surgical tray between each of the top corners.

16. The method according to claim 14, wherein the plurality of corner protectors and edge guards provide a cushion between the surgical tray and sterilization wrap.

17. The method according to claim 14, wherein each of the plurality of corner protectors includes:
(a) a base;
(b) first and second sides extending upwardly from a top of the base, each of the sides having a bottom edge, wherein a first portion of each bottom edge is connected to the base and a second portion of each bottom edge extends beyond the base such that a distal end of each side is disposed beyond the base; and
(c) wherein proximal ends of the first and second sides are joined to each other so as to define a corner, the first and second sides cooperate with the base to define three mutually perpendicular planes that define an open area configured to receive a corner of a surgical tray having a side wall with a height substantially greater than the first and second sides of the corner protector therein.

18. The method according to claim 14, wherein each of the plurality of edge guards includes:
(a) a first side having a proximal end and a distal end;
(b) a second side having a proximal end and a distal end, the proximal end of the second side being joined to the proximal end of the first side such that the first side and second side are substantially perpendicular to each other and form an elongated strip adapted to conform to an edge of the surgical tray.
